(19) United States
(12) Patent Application Publication
(10) Pub. No.: US 2011/0250632 A1
(43) Pub. Date: Oct. 13, 2011

(54) METHODS FOR CONDUCTING CELLULAR ASSAYS

(75) Inventors: Peter James Tatnell, Cardiff (GB); Stephen Game, New Milton (GB); Anne Michelle Doyle, Cardiff (GB)

(73) Assignee: GE HEALTHCARE UK LIMITED, LITTLE CHALFONT (GB)

(21) Appl. No.: 13/140,056

(22) PCT Filed: Dec. 16, 2009
(86) PCT No.: PCT/EP09/67249

§ 371 Date: Jun. 16, 2011

(30) Foreign Application Priority Data
Dec. 18, 2008 (GB) 0823056.7

(51) Int. Cl. C12N 5/071; C12Q 1/02; C12N 11/00

(52) U.S. Cl. 435/29; 435/174; 435/374

(57) ABSTRACT

The present invention relates to cryopreserved cell cultures, methods for cryopreserving cells and methods for conducting cellular assays on such cells. A cryopreserved cell culture of the invention comprises a container having at least a surface which is coated with poly-lysine and frozen cells supported on the surface.

### CROSS-REFERENCE TO RELATED APPLICATIONS

This application is a filing under 35 U.S.C. §371 and claims priority to international patent application number PCT/EP2009/067249 filed Dec. 16, 2009, published on Jul. 15, 2010 as WO 2010/079058, which claims priority to application number 0823056.7 filed in Great Britain on Dec. 18, 2008.

### FIELD OF THE INVENTION

The present invention relates to cellular assays or cell based assays and in particular to the provision of cryogenically-preserved cells for use in such assays.

### BACKGROUND OF THE INVENTION

Drug discovery, as currently practiced in the art, is a long, multiple step process. Initially this process involves identification of specific disease targets, development of an assay based on a specific target, validation of the assay, optimization and then the automation of the assay to produce a screen. High throughput screening (HTS) of compound libraries using the assay can then be carried out to identify candidate compounds, which show promise as potential drugs; these compounds are then validated and chemically optimized. The output of this process is a lead compound that goes into pre-clinical trials and, if validated, eventually into clinical trials. In this process, the screening phase is distinct from the assay development phases, and involves testing compound efficacy in living biological systems.

Historically, drug discovery is a slow and costly process, spanning numerous years and consuming hundreds of millions of dollars per drug created. Developments in the areas of genomics and HTS have resulted in increased capacity and efficiency in the areas of target identification and compound screening.

The application of HTS technologies offers the potential to address and ease the bottlenecks currently encountered in the drug discovery process. In the HTS process, drug candidates are screened for possible effects in biological systems and for the specificity of selected lead compounds towards particular targets. Primary screening has been addressed by the development of HTS assay processes and assay miniaturisation utilizing the microtitre well plate format, with 96, 384, 1536 or greater miniaturised wells, which is capable of allowing throughput levels of over 100,000 tests/day.

Many types of cell-free assays that were originally developed to measure the biochemical activity of purified proteins, mostly enzymes, could be readily converted to HTS by applying detection systems that do not require separation of the reaction product from substrate. For example, fluorescence-based techniques allow detection of enzymatic activities as well as of molecular interactions without the requirement of separation, fractionation or purification procedures (Hertzbamerg R P and Pope A J. High-throughput screening: new technology for the 21st century. Curr. Opin. Chem. Biol. 4, pp 445-451, 2000). These automated assays allow rapid screens of large compound libraries to identify so-called ‘hits’, namely compounds that show the desired effect on the biochemical activity of the specific target in the isolated in vitro system. Hits are then subjected to chemical modifications and further screening through the HTS system to select more specific and potent derivatives called ‘lead’ compounds. In the classical drug discovery process, lead compounds are subsequently tested in various in vivo assays using cellular and animal models in order to select those that may become drug candidates for clinical trials.

In the last few years, cell-based assays using engineered cells and microorganisms have become an increasingly attractive alternative to in vitro biochemical assays for HTS in the early phase of the drug discovery process. The requirements for such assays are the ability to examine a specific cellular process triggered by a defined target and a means to readily measure its output in a HTS system. The availability of an increasing number of biotechnological tools to genetically modify cells and microorganisms has allowed the development of simple read-out assays for cellular processes that can be readily applied to automated systems in HTS.

Cell-based assays have notable advantages over in vitro traditional biochemical assays. Firstly, these cellular assays do not require purification of the target protein and therefore eliminate investment of resources to gain the necessary knowledge for obtaining a biochemically active target—this advantage has become particularly important with the increasing number of proteins that can be targeted for potential drug treatment as it would indeed be difficult to set up specific biochemical assays for hundreds of new proteins for which the natural substrates remain largely unknown. Secondly, the conformation and the activity of the target protein, as well as the read-out to monitor the effect of compounds, are examined in a cellular context that most likely represents the natural physiological state more closely than in vitro assays. Thirdly, cell-based assays can immediately select against compounds that are generally cytotoxic, or that cannot permeate cellular membranes to reach intracellular targets. Thus, hit and lead compounds that are identified through cell-based assays have passed important validation steps. The availability of this information provides a head start compared to many in vitro assays and can save valuable time and costs in the development of the drug.

However, the recent growth in cell use for drug discovery, particularly for HTS, poses a number of challenges for cell providers and users; namely, batch performance variation, cell production scheduling, and capability and capacity management.

Cell lines are usually subcultured twice a week and scaled up for each assay. This subculturing and upscaling is usually repeated in cycles over a period of several months. The use of cryopreserved cells, grown in a large single batch and stored in the freezer, provides significant advantages: (a) improved consistency of cell-based assay results—once frozen, the same cell batch can be used over a long period of time, (b) increased flexibility—new assays can start at any moment when compounds arrive for testing, and (c) reduced costs—time spent to maintain cell lines in culture in parallel to drug screening activities is saved. Consequently, the use of cell culture reagents, disposables and cell culture facilities is reduced.

Cryopreservation per se has generally no effect on the pharmacology of compounds and can be applied to many cell types and assays (Guido J. R. Zaman, et al. Cryopreserved cells facilitate cell-based drug discovery. Drug Discovery Today. Volume 12, Numbers 13/14, July 2007). For example, cryopreserved, transiently transfected HepG2 cells were compared to freshly transfected HepG2 cells for use in a pregnane X receptor (PXR) transactivation assay. Assay performance was similar for both cell preparations; however, cryopreserved cells demonstrated less inter-assay variation. Validation with drugs of different PXR activation potencies and efficacies demonstrated an excellent correlation (r²>0.95) between cryopreserved and fresh cells. Cryopreservation did not change the effect of known CYP3A4 inducers that have poor cell permeability, indicating that cryopreservation had little effect on membrane permeability. In addition, cryopreserved HepG2 cells did not exhibit enhanced susceptibility to cytotoxic compounds compared to transiently transfected control cells. The use of cryopreserved cells enables this assay to run with enhanced efficiency (Zhu, Z. et al. Use of cryopreserved transiently transfected cells in high-throughput pregnane X receptor transactivation assay. Journal of Biomolecular Screening. 12, 248-254, 2007). The examples described in the literature include cell types that are widely used in drug screening, such as CHO and HEK293, and readouts such as beta-lactamase and FLIPR.

Louise Stjernborg and colleagues from AstraZeneca (Molndal, Sweden) described the use of frozen cells for the screening of compound side effects on human ERG (hERG) channel with a rubidium efflux assay. In their organization, hERG channel screening was not conducted every day but on a monthly basis, when sufficient new compounds were assembled. The use of frozen cells resulted in a significant saving of time spent on cell culture maintenance (Ding, M. et al. Application of cryopreserved cells to hERG screening using a non-radioactive Rb+ efflux assay. Assay Drug Dev. Technol. 4, 83-88, 2006.)

Thus use of cryopreserved cells in drug discovery has three advantages. Firstly, flexibility is increased, because new assays can start at any moment. Secondly, data quality is improved, as all testing results for a certain compound in a certain assay can be generated with the same batch of cells. Thirdly, working with frozen cells substantially reduces the time spent on cell culture work, in particular the maintenance of cell lines, and consequently the use of cell culture facilities, materials and disposables.

Poly-lysine is used as a non-specific attachment factor for cells and is routinely used in promoting the adhesion of certain cell types to solid substrates such as synthetic culture surfaces; glass slides electron microscopy grids etc. (Jacobson, B. et al. Plasma membrane: rapid isolation and exposure of the cytoplasmic surface by use of positively charged beads. Science 195: 302-304, 1977). It is generally used for cells, which do not normally adhere to solid surfaces (Mazia, et. al. Adhesion of cells to surfaces coated with polylysine. Applications to electron microscopy. The Journal of Cell Biology, Vol 66, 198-200, 1975). This is achieved by simply coating the solid surface (plastic, glass etc) with a solution of cationic poly-lysine. The poly-lysine moieties form electrostatic interactions with the negatively charged molecules present in the plastic and glass surfaces.

Poly-lysine however, is also used to increase the adhesion of many other non-mammalian cells and tissues in techniques ranging from traditional live cell-based analyses to immuno-histochemistry. Many examples of its use to facilitate the attachment of mammalian/non-mammalian cells are present in the scientific literature. Poly-lysine is used to attach cells derived from the all the major biological species including mammalian, insect, amphibian, fish, reptile and avian families. In addition, examples are given that include cells derived from species as diverse as bacteria, nematodes and gastropods.

Some examples of cell lines routinely cultured on poly-lysine include—HEK293 human embryonic kidney cells (Sugawara, T. et. al. A missense mutation of the Na+ channel alpha II subunit gene Na(v)1.2 in a patient with febrile and afebrile seizures causes channel dysfunction. Proc Natl Acad Sci USA. 98(11), 6384-9, 2001), MDA-231, breast cancer cell line (Yoneda, T. et. al. Inhibition of osteolytic bone metastasis of breast cancer by combined treatment with the bisphosphonate ibandronate and tissue inhibitor of the matrix metalloproteinase-2. J. Clin. Invest. 99(10), 2509-17, 1997), anterior pituitary cells (Hinuma, S, et. al. A prolactin-releasing peptide in the brain. Nature, 393(6682), 272-6, 1998), microglia MG-7 cells (Szczepanik, A M. et. al. Amyloid-beta peptide fragments p3 and p4 induce pro-inflammatory cytokine and chemokine production in vitro and in vivo. J. Neurochemistry, 77(1), 304-17, 2001), and rat primary astrocytes (Little, E B. et. al. A short segment within the cytoplasmic domain of the neural cell adhesion molecule (N-CAM) is essential for N-CAM-induced NF-kappa B activity in astrocytes. PNAS USA, 98(5), 2238-43, 2001).

GB2427411A relates to cryopreservation methods for cells derived from tissues using alginate/polysaccharide microcapsules/hydrogels. Poly-lysine is used to facilitate cell attachment to the polysaccharide. There was no mention of the use of poly-lysine for improvement in assay performance in this document.

U.S. Pat. No. 6,657,003 discloses a liquid solution for coating substrates that demonstrates long-term stability & cell adhesion properties. More particularly, it refers to a solution including a cross-linked amino acid polymer for applying to cytological specimen slides. The amino acid polymer, as employed in the invention, is selected from a neutral or basic amino acid, such as poly 1-lysine or poly 1-arginine.

WO 2005/034625 relates to a surface (such as a cell culture surface) comprising a support to which is bound a cell adhesion resistant (CAR) material and, bound to the CAR material, collagen VI or a biologically active fragment or variant thereof and, optionally, one or more other ECM proteins, or biologically active fragments of variants thereof and/or one or more polycationic polymers.

U.S. Pat. No. 5,512,474 describes cell culture surfaces of bioreactors in the field of cell biology and particularly to methods of improving the surfaces to obtain better cell attachment and cell growth. A cell culture support comprising a support material in the form of a microcarrier and comprising a supporting surface for the attachment of cells is disclosed, the surface bearing a combination comprising: a positively-charged molecule and acrylics polymerized from acrylamide or methacrylamide.

U.S. Pat. No. 5,932,473 describes a cell culture substrate coated with a composition containing a cell adhesion promoter in a salt solution. A substrate such as plastic, glass or microporous fibers is coated with a composition containing about 5 μg/ml to about 1000 μg/ml of poly-D-lysine in an 0.005 M to about 0.5 M citrate or sulfate salt solution to provide about 50 μl to about 500 μl of the composition per cm² of substrate.

Ma, M. et al., 2006 (Neuroscience Lett., 403, 84-89) described the cryopreservation of an adherent mammalian neuronal network. Dissociated spinal cord cells were attached to 35 mm Petri dishes that had been coated with two adhesion factors poly-D-lysine and laminin. The network was then embedded in collagen and loaded with the cryoprotectant trehalose prior to transfer to a freezing medium containing DMSO, FBS and culture medium. The network was stored for up to 2 months in liquid nitrogen at −196° C. Characterisation of the network post cryopreservation involved; determining cell viability using fluorescent probes (Viability/Cytotoxity kit, Molecular Probes), the expression of neuronal markers by immunocytochemistry and elucidation of synaptic vesicle recycling using the fluorescent probe FM1-43 (Molecular Probes).

Son, J. H. et al., 2004 (Biotechnology Letters, 26, 829-833) described the use of type 1 collagen coated 6-well plates and a freezing mixture containing DMSO, foetal calf serum and culture medium to facilitate the cryopreservation of rat hepatocytes. The cells were cryopreserved for only a short period of time (24 hr) at −70° C. Characterisation of the hepatocytes post cryopreservation involved determining cell viability using a simple spectrophotometric MTT assay and albumin secretion using an ELISA-based system.

Shoji R. et al., 2000 (Cytotechnology, 32, 147-155) described the cryopreservation of human hepatoma cells using 96-well plates coated with either type 1 collagen, pronectin F or fibronectin. These reagents were used to facilitate cellular attachment. Cryopreservation was achieved using hepatocyte culture medium supplemented with 10% DMSO at −85° C. These conditions maintained the cells in the best state for ˜7 days. Characterisation of the hepatocytes post cryopreservation was limited to cells stored for only 7 days and involved determining cell viability by measuring acid phosphatase activity and in-vitro cytotoxicity testing (live/dead dose response curves in response to challenge with toxic reagents).

### TECHNICAL PROBLEM

There is a requirement within the pharmaceutical industry to cryopreserve cells in containers which can be used both for storage at low temperatures and subsequently for screening purposes; thus the containers would be stored at sub-zero temperatures and simply removed from the freezer to allow the cells to thaw and used directly in HTS. Currently, cells are generally cryopreserved in vials (e.g. cryovials) and stored in these vials at low temperatures until required for testing; the cells are then reconstituted/thawed gradually, washed with buffer and transferred to culture dishes or microwell plates in readiness for testing/assay in HTS. At this time, cells cannot be cryopreserved in culture dishes or microwell plates due to both high mortality on thawing and variability in assay response. The latter is evident in the low Z-factors obtained in such assays. The ability to cryopreserve cells and conduct assays on the reconstituted cells in the same container or vessel would provide an enabling technology for the pharmaceutical industry.

Therefore, there is a need to improve the workflow by removing one or more steps in the transfer process from vial/cryovial to culture dish/microwell plate, thus simplifying the procedure to reduce cost and time. Furthermore, there is a need to improve cell viability and/or assay performance of cells, which have been cryopreserved in culture dishes or microwell plates.

The present invention addresses the above problems and provides cryopreserved cell cultures and methods for producing the same, which permit cryopreservation and assay of reconstituted cells in a single container.

### SUMMARY OF THE INVENTION

According to a first aspect of the invention there is provided a cryopreserved cell culture, comprising: a container having at least a surface, frozen cells supported on the surface, wherein the surface is coated with poly-lysine.

Poly-lysine is known to facilitate the attachment of certain cell types to surfaces but also surprisingly has been found to improve assay performance (even after prolonged cryopreservation) by a process that is independent of cell attachment.

Poly-lysine is routinely used as a molecule to increase the adhesion of mammalian cells to cell culture treated surfaces. This is achieved by simply coating the solid surface (plastic, glass etc) with a solution of cationic poly-lysine. The poly-lysine moieties may form electrostatic attractions with the negatively charged molecules present in the plastic and/or glass surfaces.

Poly-lysine is available from a number of suppliers (e.g. Sigma, P7405, >300 k or P6407, 70-150 k). Poly-lysine enhances the electrostatic interactions between negatively charged ions of the cell membrane and the culture surface. When adsorbed on to the culture surface, poly-lysine increases the number of positively charged sites available for cell binding. Polymers of both poly-D- and poly-L-lysine, and mixtures thereof, can be used to coat solid surfaces. However certain cells are able to proteolytically degrade poly-L-lysine, in this situation poly-D-lysine is generally used to prevent excessive degradation and eventual uptake of L-lysine.

Lower molecular weight poly-lysine (Mᵣ=30,000-70,000 kD) is easier to use because it is less viscous in solution, however higher molecular weight >300,000 kD provides better cell attachment per molecule. The molecular weight of poly-lysine for general use in cell biology is typically in the range of 70,000-150,000 kD.

In another aspect of the invention the cell culture container is selected from the group consisting of vessel, vial, microtitre plate and cell culture plate.

In yet another aspect of the invention the cells frozen on the surface of the cell culture are selected from the group consisting of mammalian cells, insect cells, amphibian cells, fish cells, reptile cells and avian cells. Preferably the cells are mammalian cells. More preferably, the cells are selected from the group consisting of CHO cells (Source: ECACC-85050302), HEK293 cells (Source: ATCC-CRL1573) and AD293 cells (Source: Invitrogen R705-07).

According to a second aspect of the invention, there is provided a method of cryopreserving cells in a cell culture, the method comprising the steps of:
a) adding a medium containing cells to a surface of a container to allow the cells to attach to the surface and form a cell culture
b) adding a cryopreservation medium, and
c) reducing the temperature of the cell culture to −20° C. or below;
   wherein the surface is coated with poly-lysine prior to step a).

Poly-lysine is a molecule used as a coating to enhance cell attachment to plastic and glass surfaces. It has been used to culture a wide variety of cell types, including neurons, glial cells and transfected cells. Poly-D Lysine is commonly used as a culture substrate to promote adhesion, growth, and differentiation for a variety of neuronal and transfected cell lines. Both poly-D, poly-L lysine and mixtures thereof, can be used to coat solid surfaces and traditionally functions as non-specific attachment factors for cells. One of the known functions of poly-lysine is to enhance the electrostatic interaction between negatively charged ions associated with the cell membrane and the cell culture surface. When absorbed to the cell culture surface poly-lysine increases the number of positively charged sites available for cell binding.

In another aspect of the invention the cell culture container is selected from the group consisting of vessel, vial, microtitre plate and cell culture plate.

In yet another aspect of the invention the cells frozen on the surface of the cell culture system are selected from the group consisting of mammalian cells, insect cells, amphibian cells, fish cells, reptile cells and avian cells. Preferably, the cells are mammalian cells. More preferably, the cells are selected from the group consisting of CHO cells, HEK293 cells and AD293 cells.

In another aspect of the invention the cells were stored at a temperature below −80° C. More preferably the cell culture plate was frozen to −80° C. for 16 hrs followed by transfer for longer storage at −140° C.

In a third aspect of the invention, there is provided a method for conducting a cellular assay, the method comprising the steps of: a) adding a medium containing cells to a surface of a container to allow the cells to attach to the surface and form a cell culture; b) adding a cryopreservation medium; c) reducing the temperature of the cell culture to freeze the cells; d) storing the cell culture at a temperature of less than −20° C.; e) thawing the cells by raising the temperature of the cell culture; and f) conducting a cellular assay on the cells, wherein the surface of the container is coated with poly-lysine prior to step a).

In one aspect, the method comprises adding a medium containing cells to a surface of a container to allow the cells to attach to the surface for a period of 16 hrs and form a cell culture, replacing the growth medium with cryopreservation medium (90% fetal calf serum and 10% of Dimethyl Sulphoxide (DMSO)), reducing the temperature of the cell culture to freeze the cells, storing the cell culture at a temperature of less than −20° C., then thawing the cells by raising the temperature of the cell culture, and conducting a cellular assay on the cells.

In another aspect the cell culture is stored at a temperature of −80° C.

In a further aspect, poly-lysine is selected from the group consisting of poly-D-lysine, poly-L-lysine and mixtures thereof.

In yet a further aspect the container is selected from the group consisting of vessel, vial, microtitre plate and cell culture plate. Preferably, the container is a cell culture plate.

In one aspect, the cells are adherent cells. In particular, the cells are selected from the group consisting of mammalian cells, insect cells, amphibian cells, fish cells, reptile cells and avian cells. Preferably, the cells are mammalian cells. More preferably, the cells are selected from the group consisting of CHO cells, HEK293 cells and AD293 cells.

In another aspect, the cells used in step a) are attached to one or more micro-carriers. Preferably, the micro-carriers are coated with poly-lysine.

In a yet another aspect of the invention the cell culture container is selected from the group consisting of vessel, vial, microtitre plate and cell culture plate.

### Definitions:

The following terms are to be understood in the context of the present invention:
Cellular Assay—a method or test for examining cellular processes triggered by the action of a compound and a means to measure the cellular output. Such assays are of particular use in drug screening. It will be understood by the skilled person that cell death or viability would not be included in this definition.
Cryopreserved—preserved by the use of cryopreservation (the preservation of biological tissue including intact and viable cells at cryogenic temperatures, typically at −20° C. and below).
Frozen cells—intact and viable biological cells maintained at −20° C. or below.
Cryopreservation medium—sometimes referred to as “cell freezing medium” is any medium which contains a reagent or composition which reduces cellular damage or injury during cryopreservation or freezing. Examples of such cryopreservation media include, but are not limited to, Dimethyl Sulphoxide (DMSO), foetal calf serum, glycerol, Dulbecco's Modified Eagle's Medium (DMEM), trehalose and mixtures thereof.

### Signal: Background (S:B)

This is essentially the mean of the plus agonist (stimulus) assay divided by the mean of the minus agonist (stimulus) assay.

### Signal: Noise (S:N)

This formula takes into account the standard deviations observed with both the signal and background assays. It is an indication of assay performance.

$$\frac{Mean ( plus agonist ) - Mean ( minus agonist )}{\sqrt{\begin{matrix}{( Standard deviation plus agonist )}^{2} - \\ {( Standard deviation minus agonist )}^{2}\end{matrix}}}$$

### Z-factor

This is used generally by the pharmaceutical industry to assess assay performance. In statistics, the Z-factor is a measure of the quality or power of a high-throughput screening assay. The formula includes the means and standard deviations of plus and minus agonist assays. The closer the value approaches 1 the better the performance. For cell based assays >0.40 is generally considered good, while values of 0.5 and above are considered excellent (Zhang et.al. (1999) A Simple Statistical Parameter for Use in Evaluation and Validation of High Throughput Screening Assays. (J Biomol Screen., 4 (2) 67-73).

$$1 - ( \frac{\begin{matrix}3 ( Standard deviation plus agonist ) + \\ 3 ( Standard deviation minus agonist )\end{matrix}}{( Mean plus agonist ) - ( Mean minus agonist )} )$$

Further features and advantages of the invention will become apparent from the following description of preferred embodiments of the invention, given by way of example only, which is made with reference to the accompanying drawings.

### BRIEF DESCRIPTION OF THE DRAWINGS

FIG. 1a shows a comparison of images of Chinese hamster ovary cells stably expressed in a recombinant protein consisting of a Vesicular Stomatitis Virus epitope tag-β2 adrenergic receptor-Enhanced green fluorescent protein cells (CHO VSV-β2AR-EGFP stable cells) grown on plates coated with and without poly-lysine before cryopreservation and after cryopreservation.

FIG. 1b shows a comparison of images of Human Embryonic Kidney 293-Vesicular Stomatitis Virus tag-β2 adrenergic receptor-Enhanced green fluorescent protein cells (HEK293 VSV-β2AR-EGFP stable cells) grown on plates coated with and without poly-lysine before cryopreservation and after cryopreservation.

FIG. 2 shows a comparison of the viability of cell lines (CHO VSV-β2AR-EGFP stable and HEK293 VSV-β2AR-EGFP stable) seeded on to plates coated with and without poly-lysine, determined post thawing using the Cell Titer-Glo luminescent cell Viability Assay (Promega).

FIG. 3 shows the comparison of assay performance of cryopreserved AD293 cells seeded on to plates coated with and without poly-lysine.

FIG. 4 shows Agonist (a) and Antagonist (b) dose response curve for CHO VSV-β2AR-EGFP cells

FIG. 5 shows agonist (a) and antagonist (b) dose response curve for HEK293 VSV-β2AR-EGFP cells

### DETAILED DESCRIPTION OF THE INVENTION

In a first aspect, the present invention is a cryopreserved cell culture, comprised of a container having at least a surface to which the frozen cells are supported, and wherein the surface of the cell culture is coated with a poly-lysine. The provision of pre-dispensed cryopreserved cells in cell culture systems will reduce the amount of time spent on cell culture manipulations. The cells may be either transiently or stably transfected. Processing the “ready to go” pre-frozen cells would involve simply defrosting the plates, removing the freezing medium, followed by a simple PBS wash and addition of assay medium.

Poly-lysine compounds have surprisingly been found to improve assay performance (even after prolonged cryopreservation) by a process, which is independent of cell attachment. This improvement functions by at least two separate mechanisms:
i) Promoting and maintaining HEK293 cell adherence especially after prolonged cryopreservation and through multiple washing steps. Poly-lysine mediated cell attachment is a well established phenomenon for HEK293 cells; indeed increasing cell adherence is one of main cell biological uses of poly lysine.
ii) Poly-lysine affects CHO cell line morphology (Sordel et al. Influence of glass and polymer coatings on CHO cell morphology and adhesion. Biomaterials Vol. 28, Issue 8, 1572-1584, 2007). In traditional CHO cell culture, poly lysine is not used routinely as it is considered unnecessary as CHO cells adhere to “cell culture plastic” very efficiently. However, the use of poly-lysine prior to cell cryopreservation appears to enhance assay performance without influencing cell adherence. On closer inspection of the CHO cells±poly lysine, a correlation appears to exist between the numbers of cells exhibiting a raised morphology and improved assay performance (as determined by Z-factor measurements).

Both poly-D and poly-L lysine can be used to coat solid surfaces and traditionally functions as non-specific attachment factors for cells. One of the known functions of poly-lysine is to enhance the electrostatic interaction between negatively charged ions associated with the cell membrane and the cell culture surface. When absorbed to the cell culture surface poly-lysine increases the number of positively charged sites available for cell binding. Poly-lysine is available in a range of different molecular weights e.g. Mᵣ 30,000-70,000 is less viscous in solution and therefore easier to dispense however, poly-lysine >300,000 provides more attachment sites per molecule. As a compromise the preferred poly-lysine Mᵣ is 70,000-150,000.

In some instances certain cells are able to proteolytically degrade poly-L-lysine in this situation the cells can be disrupted by excessive uptake of L-lysine and therefore poly-D-lysine should be used as the polycation.

The cell culture container is selected from the group consisting of vessel, vial, microtitre plate and cell culture plate.

The cells frozen on the surface of the cell culture are selected from the group consisting of mammalian cell, insect cell, amphibian cell, fish cell, reptile cell and avian cells. The cells are preferably mammalian cells; more preferably mammalian cells selected from the group consisting of CHO cells, HEK293 cells and AD293 cells.

The following list consists of mammalian cells that are representative of those likely to be applicable for the use of the present invention. The list is given as an example and should not be considered as limiting. In addition cells, which stably express heterologous genes, are also part of this embodiment e.g. the cell lines HEK293 and CHO stably expressed with the β2 adrenergic receptor as previously described.

a. Representative Cell Lines
HeLa Negroid cervix adenocarcinoma (human)
HEK 293 Embryonic kidney (human)
1321-N1 Brain astrocytoma (human)
U-2 OS Bone osteosarcoma cell (human)
Huh-7 Hepatoma (human)
K-562 Lymphoblast (chronic myelogenous leukaemia) (human)
HepG2 Liver hepatocellular carcinoma (human)
Hep3B Hepatocellular carcinoma (human)
BJ Foreskin fibroblast (human)
CACO2 Colorectal adenocarcinoma (human)
MCF7 Human mammary gland adenocarcinoma (human)
Swiss and NIH 3T3 Embryo fibroblast (mouse)
COS-1 and -7 SV40 transformed kidney cell line (African green monkey)
CHO (CHO-K1) Chinese Hamster Ovary cells (hamster)
   b. Representative Primary Cells
HUVEC Umbilical vein endothelial cells (human)
MHEpC Mammary epithelial cells (human)
HTEpC Tracheal epithelial cells (human)
HAOEC Aorta endothelial cells (human)
PBMC Peripheral blood mononuclear cells (human)
   c. Representative Stem/Progenitor Cells
hESC-BG01V Embryonic stem cell line (human)
ES-057BL/6 Embryonic stem cell line (mouse)
MLPC Multi lineage progenitor cells umbilical cord blood (human)
   d. Representative Carcinoma Cells
NTERA 1 and 2 Testes embryonal carcinoma cells (human)

Lower molecular weight poly-lysine (Mᵣ=30,000-70,000 kD) is easier to use because it is less viscous in solution, but at higher molecular weight >300,000 kD provides better cell attachment per molecule. The molecular weight of poly-lysine for general use in cell biology is typically in the range of 70,000-150,000 kD.

Poly-lysine treated solid surfaces have been shown to support neurite outgrowths and improve the survival of many cells derived from the central nervous system. As poly-α-lysine is a synthetic compound it generally does not stimulate a biological response in the cultured cells. As it is generated synthetically it routinely does not contain any biological impurities that can be an issue associated with other natural polymers.

Some examples of cell lines routinely cultured on poly-lysine include—HEK293 human embryonic kidney cells (Sugawara, T. et. al. A missense mutation of the Na+ channel alpha II subunit gene Na(v)1.2 in a patient with febrile and afebrile seizures causes channel dysfunction. Proc Natl Acad Sci USA. 98(11), 6384-9, 2001), MDA-231, breast cancer cell line (Yoneda, T. et. al. Inhibition of osteolytic bone metastasis of breast cancer by combined treatment with the bisphosphonate ibandronate and tissue inhibitor of the matrix metalloproteinase-2. J. Clin. Invest. 99(10), 2509-17, 1997), anterior pituitary cells (Hinuma, S, et. al. A prolactin-releasing peptide in the brain. Nature, 393(6682), 272-6, 1998), microglia MG-7 cells (Szczepanik, A M. et. al. Amyloid-beta peptide fragments p3 and p4 induce pro-inflammatory cytokine and chemokine production in vitro and in vivo. J. Neurochemistry, 77(1), 304-17, 2001), and rat primary astrocytes (Little, E B. et. al. A short segment within the cytoplasmic domain of the neural cell adhesion molecule (N-CAM) is essential for N-CAM-induced NF-kappa B activity in astrocytes. PNAS USA, 98(5), 2238-43, 2001).

Poly-L-lysine has also been used to culture two different cells as patterned co-cultures. Hyaluronic acid was used to immobilise the initial cells on a glass substrates. Subsequently poly-L-lysine was absorbed in discrete patterns on to the hyaluronic acid thereby altering the properties of the culture surface and thus promoting the adherence of a second cell type. The utility of this approach has been demonstrated to co-culture embryonic stem cells with fibroblasts (Khademhosseinin, A. et. al. Layer-by-layer deposition of hyaluronic acid and poly-L-lysine for patterned cell co-cultures. Biomaterials, 25, 3583-92, 2004)

Poly-lysine coated electron microscope specimen films have been used to visualise double and single strand DNA molecules (Williams, R C. Use of polylysine for adsorption of nuclei acids and enzymes to electron microscope specimen films. PNAS, 74(6), 2311-2315, 1977)

Bifidobacterium is a Gram-positive, non-motile, anaerobic bacteria present in the human gut flora. These bacteria have been incorporated into alginate poly-L-lysine microcapsules during microcapsules preparation in order to facilitate cryopreservation. The bacterial-loaded microcapsules are freezed-dried and subjected to long term cryo-storage. In this instance the poly-lysine does not actually function as a cryoprotectant but was used as a mean to stabilise the basic alginate structure (Cui, J H. et. al. Effect of

Additives on the Viability of Bifidobacteria Loaded in Alginate Poly-l-lysine Microparticles during the Freeze-drying Process. Arch Pharm. Res., 29(8), 707-711, 2006).

Poly-L-lysine coated plastic and glass dishes have also been used to study the processes associated with the development of the fish embryo. Fish eggs generally do not adhere to many cell-culture-treated surfaces. However, the eggs will attached to the plastic and glass dishes that have been coated with poly-lysine. Sperm is then introduced and the eggs fertilised and the resultant developmental processes monitored (Andoh, T. et. al. The use of poly-L-lysine to facilitate examination of sperm entry into pelagic, non-adhesive fish eggs. Int. J. Dev. Biol. 52, 753-7, 2008).

### EXAMPLES

The present examples are provided for illustrative purposes only, and should not be construed as limiting the scope of the present invention as defined by the appended claims. All references given below and elsewhere in the present specification are hereby included herein by reference.

1. Coating of Cell Culture Plates with Poly-D-lysine.

Poly-D-lysine hydrobromide (Sigma, P7405, >300k or P6407, 70-150k)—Poly-D-lysine is dissolved to a concentration of 100 ng per ml in sterile Phosphate buffered saline (PBS). An aliquot (50 μl) is dispensed into the wells of a cell culture 96-well plate. This is incubated for 30 min at room temperature (typically 25° C.). After this time any surplus poly-D-lysine solution is decanted and the wells washed 3-times with sterile Phosphate buffered saline (PBS) (200 μl).

A final addition of 100 μl PBS is retained to prevent drying. The poly-lysine coated plates can be stored in the fridge for several days but are routinely used within 48 hrs.

The use of poly-D-lysine is given as an example and is in no way limiting.

### 2. Cryopreservation

Cells were grown in cell culture vessels using routine cell culture techniques as described in “Cells—A laboratory Manual”, Spector D. L., Goldman R. D. and Leinwand L. A., Cold Spring Harbor laboratory press (1998). At the appropriate growth phase the cells are removed from the surface of the cell culture vessels using commercially available x1 trypsin/EDTA (Ethylenediaminetetraacetic acid) and re-suspended in the appropriate cell culture medium. Cells numbers are determined using the Chemotec Nucleocounter according to manufacturer's instructions.

Cells 20,000 or 5,000 were dispensed in volumes of 100 μl & 20 μl respectively into either 96- or 384-well poly-lysine coated Costar tissue culture treated white polystyrene cell culture assay plates (Catalogue number 3917 and 3712) respectively and allowed to attach and recover for 16 hrs. The following day the medium was removed and the cells washed twice with commercially available PBS. Cryopreservation medium composed of 90% foetal calf serum and 10% DMSO was dispensed on top of the attached cells. The edges of the cell culture plate were then sealed with parafilm (Pechiney Plastic Packages) or Whatman Laboratory Sealing film and the entire plate wrapped with Saran Barrier Wrap (Dow Chemical Company). The cell culture plate was frozen to −80° C. for 16 hrs followed by transfer for longer storage at −140° C.

### 3. Assay Performance

On resurrection, the frozen cell culture plates were removed from −140° C. and allowed to defrost. The cryopreservation medium was removed and the cells were washed twice with pre-warmed PBS. Assay medium supplemented with the appropriate agonist was then added. The cell-based assay was performed using the most appropriate method and/or commercially available kit for the reporter system employed.

i) Effect of poly-lysine on Cell Attachment and Morphology,

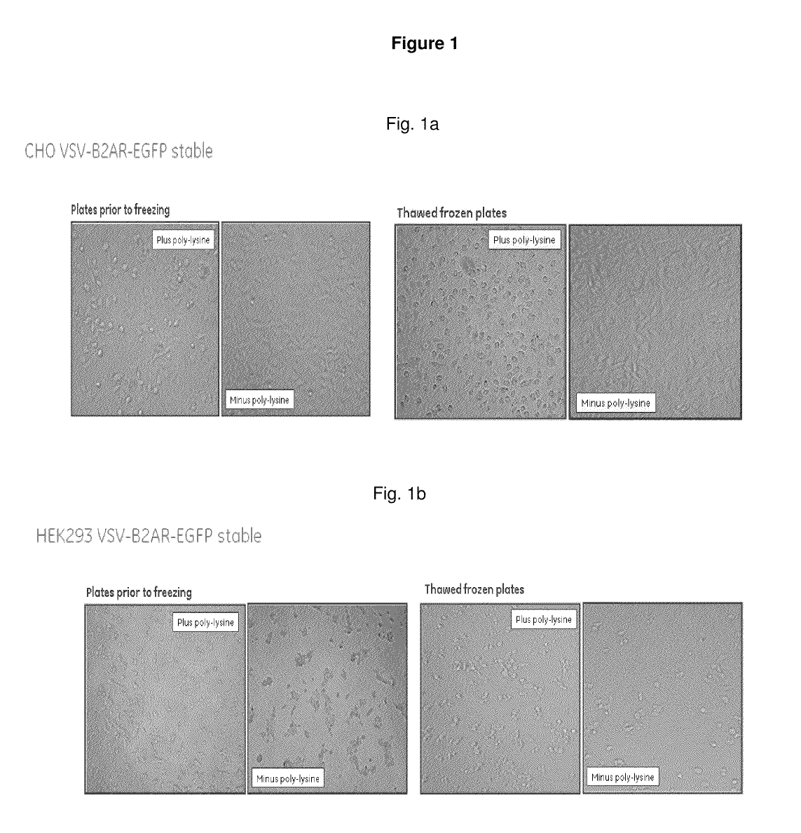

In the process to understand the effect of poly-lysine on cell attachment and morphology, images of cells grown on plates coated with and without poly-lysine before and after cryopreservation were compared (FIG. 1).

In the case of CHO VSV-β2AR-EGFP stable cell line (FIG. 1a), in the presence of poly-lysine a raised cellular morphology is observed. This morphology is absent when cells are grown in the absence of poly-lysine.

In the case of HEK293 VSV-β2AR-EGFP stable cells (FIG. 1b), poly-lysine clearly functions by promoting and maintaining cell attachment. In the absence of poly-lysine the cells are probably detaching during the cryopreservation and washing steps.

ii) Effect of poly-lysine on Viability of Cell Lines

The viability of cell lines seeded on to poly-lysine coated plates was determined post-thawing using the Cell Titer-Glo luminescent cell viability assay (Promega).

Cells were seeded into 96-well plates (±poly-lysine), cryopreserved and then resurrected.

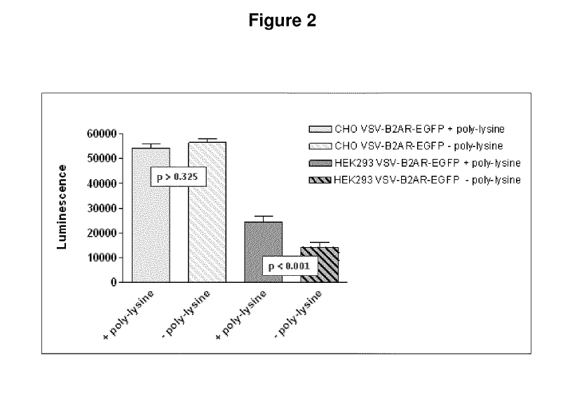

As can be seen from FIG. 2, in the case of the CHO VSV-β2AR-EGFP stable cell line, no significant difference was found in cell viability when CHO cells were plated with or without poly-lysine, indicating that poly-lysine does not promote cell attachment or improve cryopreservation.

However, in the case of the HEK293 VSV-β2AR-EGFP stable cell line, the presence of poly-lysine significantly improves luminescence signal generated. These data indicate, together with the cell image data, that poly-lysine supports and maintains HEK293 cell attachment during the cryopreservation and washing procedures.

iii) Effect of poly-lysine on Cell Assay Performance

Cells were seeded at 20,000 cells per well in 96-well plate with (+) and without (−) poly-lysine and allowed to attach overnight, growth medium was removed and freezing medium added (90% foetal calf serum and 10% DMSO). The plates were sealed and cryopreserved. As controls, cells were also cryopreserved using the traditional “Cryovial” preservation system. (Cells—A laboratory Manual, Spector D. L., Goldman R. D. and Leinwand L. A., Cold Spring Harbor laboratory press (1998).

To thaw the cells contained within the cryovial, the cryopreservation medium was diluted by cell-specific growth medium and the suspension was centrifuged (1,000×g for 5 min). The resultant cell pellet was re-suspended in fresh growth medium and the cells were dispensed into the wells of a 96-well plate ((+) and (−) poly-lysine coating), at 20,000 cells per well. The cells were incubated at 37° C., 5% CO₂ for 16 hr to facilitate cell attachment prior to assay.

On assaying, the plates were removed from the freezer and defrosted. Freezing medium was removed and cells were washed with PBS and medium containing the β2AR agonist isoproterenol was added. Increases in the cAMP levels upon GPCR activation was monitored using the DiscoverX's HITHUNTER™ cAMP II and the LEADSEEKER™ Instrument (GE Healthcare). Adenylate cyclase measurements using forskolin was used as a control (not shown).

In the experiments described both HEK293 and CHO cells stably transfected with the β2 AR were challenged with either the β2AR agonist isoproterenol or antagonist isopropranolol. On exposure to the agonist, intracellular cAMP concentrations rise and these can be monitored by a range of commercially available luminescent kits such as the HITHUNTER™ cAMPII kit (DiscoveRx) in combination with the LEADSEEKER™ Instrument platform (GE Healthcare).

As a control, rise in intracellular cAMP concentrations was also separately monitored when the ubiquitously expressed adenylate cyclase activity was challenged with its respective agonist forskolin. In the AD293 cell line only the adenylate cyclase activity was monitored.

As the DiscoveRx assay systems are designed to monitor intracellular cAMP concentrations the assay medium is supplemented with the general phosphodiesterase inhibitor isobutylmethylxanthine (IBMX) at a final concentration of 1 mM.

Isoproterenol assay—Isoproterenol (Sigma I2760) was dissolved in water to a stock concentration of 100 mM. This was then diluted to generate a final isoproterenol concentration range in the assay medium ranging from 200 μM-1 pM.

Forskolin assay—Forskolin (Sigma F6886-10 mg) was dissolved in DMSO to a stock concentration of 10 mM. This was then serially diluted using half-log dilutions to generate a final forskolin concentration range in the assay medium ranging from 316 μM-3.16 nM.

Propranolol assay—Propranolol (Sigma P8688) was dissolved in water to a stock concentration of 100 mM. This was then diluted to generate a final propranolol concentration range in the assay medium ranging from 200 μM-1 pM. The propranolol dose response curve was performed in the presence of 20 nM isoproterenol.

Non-agonist controls were also performed in which assay medium was added to the cells minus agonist or antagonist.

DiscoveRx HITHUNTER™ cAMP II assay—This was performed according to the manufacturer's instructions and briefly involved the following. On resurrection from storage at −140° C., cells frozen on cell culture treated 96-well plates were allowed to thaw. On defrosting the cryopreservation medium was removed and the cells washed twice with PBS. Assay medium supplemented with 1 mM IBMX and the appropriate agonist and/or antagonist was then added.

Typically for an assay performed in a 96-well cell culture treated plate 20,000 cells were used. On addition of assay medium to the cells the assay plate was incubated for 30 min at 37° C., after which 40 μl of the DiscoveRx cAMP II ED/substrate mixture was added. This consists of 1 part cAMP II ED reagent with 1 part Substrate Working Solution (Substrate Working Solution consisted of 1 part Galacton-Star, 5 parts Emerald II and 19 parts Substrate Diluent). Assay plates were swirled to ensure efficient cell coverage. An equal volume of EA-Ab/Lysis mix (40 μl) was then added. This consists of 1 part cAMP II EA-Ab reagent and 1 part cAMP II lysis buffer. The assay plates were incubated at room temperature for 4 hrs and the cAMP concentration determined by monitoring the generation of the luminescent signal using the LEADSEEKER™ Instrument platform.

Assays performed in 384-well cell culture treated plates are as described above except that only 5,000 cells are dispensed per well and a 50% reduction in the volumes of the DiscoveRx cAMP II ED/Substrate mixture and EA-AB/Lysis mix is used (i.e. reduced to 20 μl).

In the case of CHO VSV-β2AR-EGFP cells, in the absence of poly-lysine, the assay performance of plates frozen for more than 4 weeks is reduced in terms of the S/B, S/N and especially Z-factor compared to cryovial and plates coated with poly-lysine. Comparable assay performance is exhibited by both the (i) cryovial preserved cells when assayed on plates coated and uncoated with poly-lysine, and (ii) cells cryopreserved directly and assayed on plates coated with poly-lysine. For cells cryopreserved in cryovials and subsequently resurrected and transferred to microtitre plates for assay, poly-lysine appears to have little influence on assay performance, as can be seen from Table 1.

TABLE 1
CHO VSV-β2AR-EGFP (±1 μM Isoproterenol)

| | Plus poly-lysine | | | Minus poly-lysine | | |
|---|---|---|---|---|---|---|
| Weeks | S:B | S:N | Z-Factor | S:B | S:N | Z-Factor |

Frozen plate format

| | | | | | | |
|---|---|---|---|---|---|---|
| 1 | 8.0 | 4.9 | 0.34 | 5.0 | 1.8 | −0.83 |
| 2 | 6.7 | 5.9 | 0.43 | 4.9 | 2.8 | −0.19 |
| 4 | 8.5 | 10.6 | 0.67 | 7.1 | 3.3 | 0.01 |

Cryovial Storage

| | | | | | | |
|---|---|---|---|---|---|---|
| 1 | 15.5 | 8.8 | 0.64 | 16.6 | 8.2 | 0.61 |
| 2 | 7.9 | 9.6 | 0.59 | 6.6 | 12.8 | 0.74 |
| 4 | 8.3 | 11.0 | 0.70 | 7.9 | 23.7 | 0.84 |

In the case of HEK293 VSV-β2AR-EGFP cell line, a positive effect of poly-lysine on assay performance was observed in both cryopreservation and assay systems i.e (i) cryopreserved and assayed on frozen plates, and (ii) cryopreserved in cryovials and transferred to plates for assay. (Table 2). In the absence of poly lysine the assay performance of resurrected cells is poor.

TABLE 2
HEK293 VSV-β2AR-EGFP (±1 μM Isoproterenol)

| | Plus poly-lysine | | | Minus poly-lysine | | |
|---|---|---|---|---|---|---|
| Weeks | S:B | S:N | Z-Factor | S:B | S:N | Z-Factor |

Frozen plate format

| | | | | | | |
|---|---|---|---|---|---|---|
| 1 | 8.4 | 5.3 | 0.31 | 2.4 | 1.0 | −2.6 |
| 2 | 11.9 | 10.3 | 0.66 | 1.9 | 0.4 | −7.7 |
| 15 | 4.8 | 11.4 | 0.67 | 1.2 | 1.7 | −1.6 |

Cryovial Storage

| | | | | | | |
|---|---|---|---|---|---|---|
| 1 | 9.4 | 4.2 | 0.27 | 2.2 | 1.5 | −1.3 |
| 2 | 4.7 | 5.7 | 0.43 | 1.2 | 0.9 | −3.1 |
| 15 | N/A | N/A | N/A | N/A | N/A | N/A |

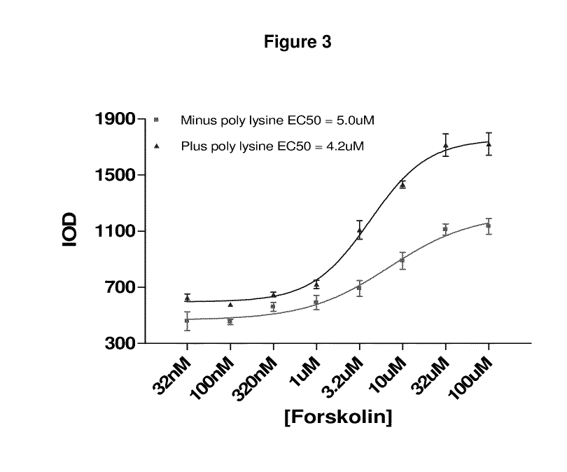

iv) Poly-lysine has a Beneficial Effect on the Assay Performance of Cryopreserved AD293 Cells (FIG. 3 and Table 3). These are HEK293 Cells that have been Engineered to Exhibit Improved Properties of Adherence.

TABLE 3

| Plus poly-lysine | | | Minus poly lysine | | |
|---|---|---|---|---|---|
| S:B | S:N | Z-factor | S:B | S:N | Z-factor |
| 3.0 | 7.1 | 0.53 | 2.5 | 5.3 | 0.33 |

As can be seen from these data, poly-lysine not only significantly increases the magnitude of the response of the AD293 cells to forskolin stimulation (FIG. 3 & Table 3) but also produces an improved Z-factor, indicative of a better assay performance.

### CHO VSV-β2AR-EGFP—Agonist and Antagonist Dose Response Curve

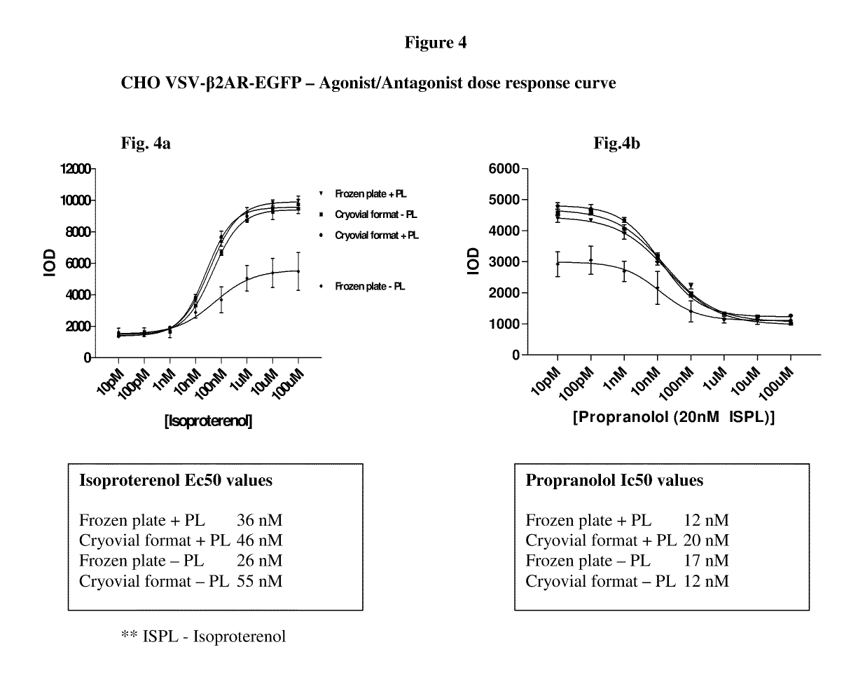

Isoproterenol E_{C}50 values—Comparable assay performance is exhibited for assays performed using cells that had either been i) cryopreserved using the traditional cryovial system (and subsequently transferred and assayed in ±poly lysine coated assay micro-titer plates) or ii) cryopreserved and assayed directly on poly-lysine coated micro-titer plates (FIG. 4a).

When cells were cryopreserved using the micro-titre format in the absence of poly-lysine, a poorer assay performance is observed. Increased signal variation in response to agonist stimulation is apparent and the magnitude of agonist induced response is decreased. Interestingly, all assay exhibit comparable E_{C}50 values in response to the agonist isoproterenol. Therefore all cells appear to be generating an appropriate agonist induced response; however, the magnitude of the response is lower when cells are cryopreserved on plates in the absence of poly-lysine.

Propranolol I_{C}50 values—A similar trend is observed for assays performed using the β2AR antagonist propranolol (performed in the presence of 20 nM isoproterenol (ISPL)). Reduced performance is observed in only those assays in which the CHO VSV-β2AR-EGFP cells are cryopreserved directly into the micro-titre plates in the absence of poly-lysine. On again comparable I_{C}50 results are observed. (FIG. 4b).

As previously shown, poly-lysine does not affect CHO cell viability or attachment. Therefore these data indicate that the presence of poly-lysine improves the assay performance of only those cells cryopreserved directly into 96-well micro-titre plates. The performance of these cells is equivalent to that demonstrated by cells cryopreserved in cryovials and subsequently transferred into assay plates. Therefore cells cryopreserved directly in poly-lysine coated plates that are also suitable for use as assay plates reduces the amount of time and manual manipulations required.

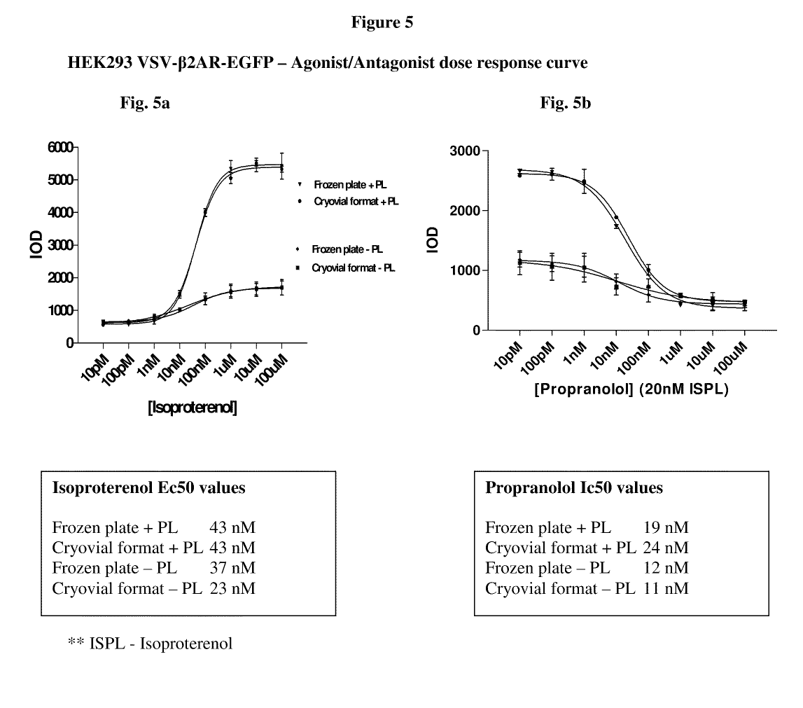

### HEK293 VSV-β2AR-EGFP—Agonist and Antagonist Dose Response Curve (FIG. 5)

Isoproterenol E_{C}50 values—Comparable assay performance is exhibited for only those assays in which the cells have been seeded on to poly-lysine coated micro-titre plates. This includes i) cell cryopreserved in cryovials and subsequently transferred to poly-lysine coated plates for assay and ii) cell cryopreserved directly in to poly-lysine coated plates (FIG. 5a)

HEK293 cells traditionally require poly-lysine to facilitate cell attachment in micro-titre plates. HEK293 cells seeded onto un-coated plates and subsequently cryopreserved exhibited a reduced number compared to the same cells cryopreserved on poly lysine coated plates. Therefore one of the properties of poly-lysine that improves HEK293 cell assay performance is related to enhanced cell attachment.

When cells were cryopreserved using the micro-titre format in the absence of poly-lysine (and even when cryopreserved in cryovials and transferred to non-coated plates), a poorer assay performance is observed. Once again, increased signal variation in response to agonist stimulation is apparent and the magnitude of agonist induced response is decreased.

A comparable E_{C}50 value in response to the agonist isoproterenol is exhibited by all assays irrespective of conditions and therefore all cells are exhibiting an appropriate response; however the magnitude of the response is lower when cells are either cryopreserved (and assayed on plates) in the absence of poly-lysine.

Propranolol I_{C}50 values—A similar trend is observed for assays performed using the β2AR antagonist propranolol (performed in the presence of 20 nM ISPL). Reduced performance is observed in only those assays in which the HEK293 cells are assayed in microtitre plates in the absence of poly-lysine irrespective of the cryopreservation medium. Once again comparable Ic50 results are observed for all assays and only the magnitude and variation is different in ±poly lysine coated plates (FIG. 5b).

These data indicate that the presence of poly-lysine improves assay performance of not only cells cryopreserved directly in 96-well micro-titre plates but also of these preserved in cryovial and subsequently transferred to poly lysine coated assay plates. The performance of HEK293 cells cryopreserved directly into poly-lysine coated plates is equivalent to that demonstrated by cells cryopreserved in cryovials and subsequently transferred into assay plates. The direct cryopreservation of cells onto plates that can subsequently be used in assays reduces both the amount of time and manual manipulations required.

These data further demonstrate that pre-coating 96-well micro-titre plates with poly-lysine prior to cryopreservation improves cellular assay performance on thawing. An additional advantage is that it also reduces the amount of time and manual manipulations required.

While preferred illustrative embodiments of the present invention are described, one skilled in the art will appreciate that the present invention can be practiced by other than the described embodiments, which are presented for purposes of illustration only and not by way of limitation. The present invention is limited only by the claims that follow.

1. A cryopreserved cell culture, comprising:
a container having at least a surface;
frozen adherent cells supported on said surface; and
cryopreservation medium;
wherein the surface is coated with poly-lysine.

2. (canceled)

3. The cell culture of claim 1, wherein said container is selected from the group consisting of vessel, vial, microtitre plate and cell culture plate.

4-5. (canceled)

6. The cell culture of claim 1, wherein the cells are mammalian cells.

7. (canceled)

8. A method of cryopreserving cells in a cell culture, said method comprising the steps of:
a) adding a medium containing adherent cells to a surface of a container to allow said cells to attach to said surface and form a cell culture;
b) adding a cryopreservation medium; and
c) reducing the temperature of said cell culture to −20° C. or below;
characterized by coating the surface with poly-lysine prior to step a).

9. (canceled)

10. The method of claim 8, wherein the surface is coated by washing the surface with a solution of poly-lysine.

11. The method of claim 8, wherein the container is selected from the group consisting of vessel, vial, microtitre plate and cell culture plate.

12-13. (canceled)

14. The method of claim 8, wherein the cells are mammalian cells.

15. (canceled)

16. The method of claim 8, further comprising the step of storing the cell culture at a temperature below −80° C.

17. A method for conducting a cellular assay, said method comprising the steps of:
a) adding a medium containing adherent cells to a surface of a container to allow said cells to attach to said surface and form a cell culture;
b) adding a cryopreservation medium;
c) reducing the temperature of said medium to freeze said cell culture;
d) storing the cell culture at a temperature of less than −20° C.;
e) thawing said cells by raising the temperature of the cell culture; and
f) conducting a cellular assay on the cells,
characterized by coating the surface of said container with poly-lysine prior to step a).

18. The method of claim 17, wherein the cell culture is stored at a temperature of less than −80° C.

19. (canceled)

20. The method of claim 17, wherein the container is selected from the group consisting of vessel, vial, microtitre plate and cell culture plate.

21. (canceled)

22. The method of claim 17, wherein the cells of step a) are attached to one or more micro-carriers.

23. The method of claim 22, wherein said one or more micro-carriers are coated with poly-lysine.

24. (canceled)

25. The method of claim 17, wherein the cells are mammalian cells.

26. (canceled)
